(19) United States
(12) Patent Application Publication
(10) Pub. No.: US 2014/0194973 A1
(43) Pub. Date: Jul. 10, 2014

(54) SAC LINER FOR ANEURYSM REPAIR

(71) Applicant: TriVascular, Inc., Santa Rosa, CA (US)

(72) Inventor: Michael V. Chobotov, Santa Rosa, CA (US)

(73) Assignee: TriVascular, Inc., Santa Rosa, CA (US)

(21) Appl. No.: 14/151,195

(22) Filed: Jan. 9, 2014

Related U.S. Application Data
(60) Provisional application No. 61/750,848, filed on Jan. 10, 2013.

(51) Int. Cl. A61F 2/07

(52) U.S. Cl. CPC A61F 2/07
USPC 623/1.15

(57) ABSTRACT

Systems and methods for managing aneurysms provide additional support to an aneurysmal wall by disposing a flexible vascular liner against or in close proximity to the aneurysmal wall. The liner is flexibly expansive to conform to the wall of the aneurysm. The liner inhibits failure of the aneurysmal wall. The liner may also inhibit further growth in diameter of the aneurysm. Aneurysms in single arteries or near branched arteries may be supported by a flexible vascular liner.

### CROSS-REFERENCE TO RELATED APPLICATIONS

This application claims the benefit of U.S. Provisional Application No. 61/750,848, filed Jan. 10, 2013, the content of all of which is incorporated herein by reference.

### FIELD OF THE INVENTION

The present invention relates to endovascular liner systems and methods for managing aneurysms. In particular, the present invention relates to endovascular liner systems and methods for providing additional support to aneurysmal walls by disposing a vascular liner against or in close proximity to aneurysmal walls.

### BACKGROUND OF THE INVENTION

The present invention relates to a system for the treatment of disorders of the vasculature, particularly aneurysms. An aneurysm is a medical condition indicated generally by an expansion and weakening of the wall of an artery of a patient. Aneurysms can develop at various sites within a patient's body. Thoracic aortic aneurysms (TAAs) or abdominal aortic aneurysms (AAAs) are manifested by an expansion and weakening of the aorta. AAAs and TAAs are serious and life threatening conditions for which intervention is generally indicated. Existing methods of treating aneurysms include invasive surgical procedures with graft replacement of the affected vessel or body lumen or reinforcement of the vessel with a graft.

Surgical procedures to treat aneurysms can have relatively high morbidity and mortality rates due to the risk factors inherent to surgical repair of this disease, as well as long hospital stays and painful recoveries. Due to the inherent risks and complexities of surgical repair of aortic aneurysms, endovascular aneurysm repair, or EVAR, has become a widely used alternative therapy, most notably in treating AAAs. Early work in this field is exemplified by Lawrence, Jr. et al. in “Percutaneous Endovascular Graft: Experimental Evaluation”, Radiology (May 1987) and by Mirich et al. in “Percutaneously Placed Endovascular Grafts for Aortic Aneurysms: Feasibility Study,” Radiology (March 1989). Commercially available endoprostheses for the endovascular treatment of AAAs include the Endurant® stent-graft system manufactured by Medtronic, Inc. of Minneapolis, Minn., the Zenith® stent-graft system sold by Cook, Inc. of Bloomington, Ind., the PowerLink® stent-graft system manufactured by Endologix, Inc. of Irvine, Calif., and the Excluder® stent-graft system manufactured by W.L. Gore & Associates, Inc. of Newark, Del. A commercially available stent-graft for the treatment of TAAs is the TAG™ system manufactured by W.L. Gore & Associates, Inc.

Typically, an endovascular stent-graft provides for a flow path for blood through an internal lumen of the stent- graft while also attempting to isolate the wall of the aneurysm from the flow of any blood. Such an endovascular approach has a common feature to the surgical approach in that aneurysmal wall tissue is removed from blood flow path(s). In this regard, endovascular stent-grafts are provided as generally tubular structures having sufficient wall strength and rigidity to maintain a generally tubular shape, including bifurcated tubular shapes, after deployment.

One area of concern with the current endovascular approach for treating aneurysms is endoleaks. (See, e.g., Bashir, Mustafa R., et al., “Endoleak After Endovascular Abdominal Aortic Aneurysm Repair: Management Strategies According to CT Strategies”, American Journal of Roentgenology (AJR):192, April 2009, W178-W186; Kinney, Thomas B., et al, “Stent grafts for abdominal and thoracic aortic disease”, Applied Radiology, March 2005, 9-19). An endoleak is characterized by persistent blood flow within the aneurysm sac following EVAR. Normally the aortic stent-graft used for EVAR excludes the aneurysm from the circulation by providing a conduit for blood to bypass the sac. Some categories of endoleaks include: Type I (e.g., leak at graft attachment site), Type II (e.g., aneurysm sac filling via branch vessel), Type III (e.g., leak through defect in graft), Type IV (e.g., leak through graft fabric as a result of graft porosity) and Type V (e.g., endotension or continued expansion of aneurysm sac without demonstrable leak on imaging). Type II Endoleaks have been reported as being the more prevalent endoleak type with EVAR procedures. (See e.g., Bashir et al., supra).

Approaches to address the endoleak concerns with EVAR procedures have been proposed. For example, WO 01/201108 A1 describes an implant for treating aneurysms by substantially filling the aneurysmal sac. Filling material include foam, sponge or other expandable material for inflating the implant. The implant may contain nitinol wires or stents, and the graft may be formed of polytetrafluoroethylene (PTFE). US 2009/0210048 describes a braided stent-graft having a self-expanding bulbous section which may be disposed within the aneurysmal sac and/or conforming to the aneurysmal sac. The stent-graft may be a single layer or may be dual layered. US 2013/0289690 A1 also describes a braided stent or stent-graft having a preset shape via computerized tomography that substantially matches the shape of the aneurysm. In both cases the stent, typical shape memory metal, engages or is proximally disposed towards the aneurysmal wall. US 2011/0257725 describes a stent-graft having an inflatable outer chamber or layer which fills the aneurysmal sac to conform to the shape of the aneurysmal sac. The chamber is described as being inflatable in situ by action or pressure of the patient's blood. US 2008/0294237 and US 2008/0188923 describe a stent-graft system having a graft liner disposed between proximal and distal stents. Upon deployment, the graft liner “expands” and “conforms” to the wall of the aneurysmal sac by the action of blood pressure. The graft liner includes an inner layer and an outer layer with an absorbent therein between. The absorbent absorbs body fluids and expands to form a “strengthened” liner to provide a bypass of the aneurysm wall. US 2004/0098096 describes a stent-graft system having a dual layered graft disposed between proximal and distal stents. Upon deployment, the outer graft liner conforms to the shape of the aneurysmal sac and is disposed therein. The space between the inner and outer graft layers may be filled with a “polymerizable” fluid via a catheter. Alternatively, the inner graft layer may be blood permeable to allow entry of blood between the space between the inner and outer graft layers. U.S. Pat. No. 8,231,665; U.S. Pat. No. 8,236,666; US 2006/0292206 and US 2007/0061005 describe a deployable balloon which upon deployment conforms to aneurysmal sac wall. The balloon is fillable with a biocompatible fluid, which may be curable. U.S. Pat. No. 7,530,988; US 2006/0212112; US 2007/0150041; US 2007/0162106; US 2009/0198267; US 2009/0318949; US 2009/0319029 and US 2011/0276078 describe stent-graft structures having an inflatable outer graft membrane disposed over an inner graft layer.

Such approaches include complicated deployment techniques; e.g., deployment of fluids, foam, sponge or other expandable material for inflating implant portions with the aneurysmal sac, or specialized stent-grafts, typically having braided nitinol stent wires, to engage the aneurysmal wall. Such approaches also fail to recognize that even aneurysmal walls have healthy, relatively healthy or viable tissue that is capable of providing some support against aneurysmal failure.

### SUMMARY OF THE INVENTION

The present invention is directed to systems and methods for treating and managing aneurysms. The present invention is a new approach to aneurysm treatment and management. Rather than mimicking traditional open repair by endovascular insertion of an internal bypass graft through the aneurysm, the present invention in one embodiment inserts a “liner graft” which is sized diametrically to reach the aneurysm wall, augment the aortic wall and prevent leaks in the event of a local structural weakening/failure of the wall. Other therapies to date have excluded the aortic wall/aneurysm sac completely, yet these structures are nearly and sometimes completely adequate for blood flow/pressure containment (for prophylactic aneurysm repair). The liner approach of the present invention can include adding a thin, strong layer of material (e.g. PTFE) to the interior surface of the aorta to reinforce the wall and prevent leaks in any weak area. The ends of the liner may be held in place with a bare stent-like structure (frame) that only attaches to the liner at its ends. Upon deployment, the liner of the present invention will be pushed to the wall/thrombus by the hemodynamic pressure gradient once seals are established at its ends, and it will also avoid or prevent Type 2 endoleaks in the process without having to separately fill the sac with other materials. A tubular section of the aorta (e.g. TAA or iliac aneurysm) may be treated with one such device.

In some embodiments, the nominal blood flow lumen may be acutely maintained by a bare stent structure, and the space exterior to the stent would nominally fill with clot over time. A tubular section of the aorta (e.g., TAA or iliac aneurysm) may be treated with one such device, while a bifurcated aneurysm can be treated with two such tubular devices, each inserted from their respective femoral/iliac access paths. Back-to-back D-shaped cross sections may be used proximally in the bifurcated case. In one embodiment, an inflatable sealing ring could be used at the end(s) of the prosthesis. The prosthesis may be sized such that the free size of the liner is greater than that of the lesion. Another embodiment may use less than fully sintered PTFE to allow the liner to stretch under the pressure gradient to engage the wall. Another embodiment may use more than one layer of various types of PTFE and/or other materials of various types in a composite-type structure.

Embodiments of the present invention may treat a wide range of anatomies, and very low profiles may be achieved since the mechanical property requirements (wall strength and fixation) of the liner and anchor and/or frame are less stringent than those of a typical graft or stent-graft that is designed to carry loads by itself without little contribution from the native anatomy.

In one embodiment, an endovascular system for aneurysm management comprises a vascular liner having a proximal open end at a proximal liner portion and an opposed distal open end at a distal liner portion defining an open liner lumen having a liner wall disposed there between and further defining a medial liner portion disposed between the proximal liner portion and the distal liner portion; a proximal anchor associated with the proximal liner portion; and a distal anchor associated with the distal liner portion; wherein the liner wall of the medial liner portion is an unitary liner wall not having spaced-apart layers or membranes thereat and not having re-enforcing filaments, strands or stent portions thereat; and wherein the liner wall of the medial liner portion is flexibly expansive to conform to a wall of an aneurysm to provide additional strength to aneurysmal tissue at the aneurysm wall. The liner wall of the medical liner portion may comprise an extruded, non-textile polymeric material. The extruded, non-textile polymeric material may comprise a material selected from the group consisting of polytetrafluoroethylene, expanded polytetrafluoroethylene having a node and fibril structure, expanded porous polytetrafluoroethylene not having a node and fibril structure, and combinations thereof. The liner wall of the medial liner portion may comprise a plurality of layers of extruded, non-textile polymeric material laminated together to provide the unitary liner wall. The liner wall of the medial liner portion may comprise a plurality of layers of extruded, non-textile polymeric material sintered together to provide the unitary liner wall. The liner wall of the medial liner portion may have a wall thickness of about 0.005 inches or less than about 0.005 inches, of about 0.0012 inches or less than about 0.0012 inches, of about 0.0007 inches or less than about 0.0007 inches, and of about 0.0005 inches or less than about 0.0005 inches, and the like. The liner wall of the medial liner portion may be flexible to expand to a bulbous shape to substantially conform to the shape of the aneurysmal wall. The liner wall of the medial liner portion may have a bulbous shape to generally conform to the shape of the aneurysmal wall. The liner wall of the medial liner portion may be crimped. The proximal anchor may be securably disposed at the proximal liner portion. The proximal anchor may be securably affixed to the proximal liner portion. The distal anchor may be securably disposed at the distal liner portion. The distal anchor may be securably affixed to the distal liner portion. The medial liner portion may include a medial stent disposed between the proximal and distal anchor, where the liner wall of the medial liner portion is not securably affixed to substantial portions of the medial stent.

The proximal portion of the assembly may be D-shaped or conform approximately to a D-shape following deployment. The endovascular system may further comprise a second vascular liner having a proximal open end at a proximal liner portion and an opposed distal open end at a distal liner portion defining an open liner lumen having a liner wall disposed there between and further defining a medial liner portion disposed between the proximal liner portion and the distal liner portion; a second proximal anchor associated with the proximal liner portion of the second vascular liner; and a second distal anchor associated with the distal liner portion of the second vascular liner; wherein the liner wall of the medial liner portion of the second vascular liner is an unitary liner wall not having spaced-apart layers or membranes thereat and not having re-enforcing filaments, strands or stent portions thereat; wherein the liner wall of the medial liner portion of the second vascular liner is flexibly expansive to conform to a wall of an aneurysm to provide additional strength to aneurysmal tissue at the aneurysm wall to inhibit failure of the aneurysm wall; wherein the proximal liner portion of the second vascular liner is D-shaped or conforms approximately to a D-shape following deployment; and wherein the D-shaped portion of the vascular liner and the D-shaped portion of the second vascular liner are complimentary such that the proximal liner portions of the vascular liner and the second vascular liner are deployable within a main artery proximal to the aneurysm, the distal portion of the vascular liner is deployable with a first branched artery distal of the aneurysm and the distal portion of the second vascular liner is deployable with a second branched artery distal of the aneurysm.

In an another embodiment, a method for treating an aneurysm comprises providing a vascular system comprising: a vascular liner having a proximal open end at a proximal liner portion and an opposed distal open end at a distal liner portion defining an open liner lumen having a liner wall disposed there between and further defining a medial liner portion disposed between the proximal liner portion and the distal liner portion; a proximal anchor associated with the proximal liner portion; and a distal anchor associated with the distal liner portion; wherein the liner wall of the medial liner portion is a unitary liner wall not having spaced-apart layers or membranes thereat and not having re-enforcing filaments, strands or stent portions thereat; and deploying the vascular liner system such that the proximal anchor is disposed proximally beyond an aneurysm and such that the distal anchor is disposed distally beyond the aneurysm; and expanding the liner wall of the medial liner portion to allow the liner wall of the medial liner portion to conform to a wall of an aneurysm to provide additional strength to aneurysmal tissue at the aneurysm wall. The expansion of the liner may be performed by inflation of a balloon within its lumen. After the liner wall of the medial liner portion is deployed to the wall of the aneurysm, the vascular system inhibits failure of the aneurysmal wall and/or inhibits further growth in diameter of the aneurysm.

The proximal portion of the assembly may be is D-shaped. The method may further comprise providing a second vascular liner having a proximal open end at a proximal liner portion and an opposed distal open end at a distal liner portion defining an open liner lumen having a liner wall disposed there between and further defining a medial liner portion disposed between the proximal liner portion and the distal liner portion; providing a second proximal anchor associated with the proximal liner portion of the second vascular liner; and providing a second distal anchor associated with the distal liner portion of the second vascular liner; wherein the liner wall of the medial liner portion of the second vascular liner is an unitary liner wall not having spaced-apart layers or membranes thereat and not having re-enforcing filaments, strands or stent portions thereat; wherein the proximal liner portion of the second vascular liner is D-shaped; and deploying the D-shaped portion of the vascular liner and the D-shaped portion of the second vascular liner within a main artery proximal to the aneurysm such that the D-shaped portions are complimentary to provide flow paths of blood through the vascular liner and the second vascular liner without a substantial flow path of blood to the aneurysm; deploying the distal portion of the vascular liner within a first branched artery distal of the aneurysm; deploying the distal portion of the second vascular liner within a second branched artery distal of the aneurysm; and expanding the liner wall of the medial liner portion of the second vascular liner to allow the liner wall of the medial liner portion of the second vascular liner to conform to a wall of the aneurysm to provide additional strength to aneurysmal tissue at the aneurysm wall.

### BRIEF DESCRIPTION OF THE FIGURES

FIG. 1 is an elevational view of a sac liner assembly useful for managing, for example, thoracic aortic aneurysms according to the present invention.

FIG. 2 depicts a close up view of a proximal anchor member and connector ring of the sac liner assembly of FIG. 1.

FIG. 3 depicts a sac liner assembly of FIG. 1 deployed in, for example, a thoracic aortic aneurysm.

FIGS. 4 through 6 depict further details of sac liner assembly of the FIG. 1 according to the present invention.

FIGS. 7 through 10 depict sac liner assemblies useful for treating aneurysms near branched lumens, for example, abdominal aortic aneurysms, according to the present invention.

FIG. 11 depict the sac liner assemblies of FIGS. 7 through 10 after deployment in an abdominal aortic aneurysm according to the present invention.

FIGS. 12 and 13 depict additional embodiments of sac liner assemblies according to the present invention.

### DETAILED DESCRIPTION OF THE INVENTION

Embodiments of the invention are directed generally to methods and devices for treatment of fluid flow vessels with the body of a patient. Treatment of blood vessels is specifically indicated for some embodiments, and, more specifically, treatment of aneurysms, such as, but not limited to, thoracic aortic aneurysms and abdominal aortic aneurysms. The present invention provides various endovascular assemblies for treatment of blood vessels.

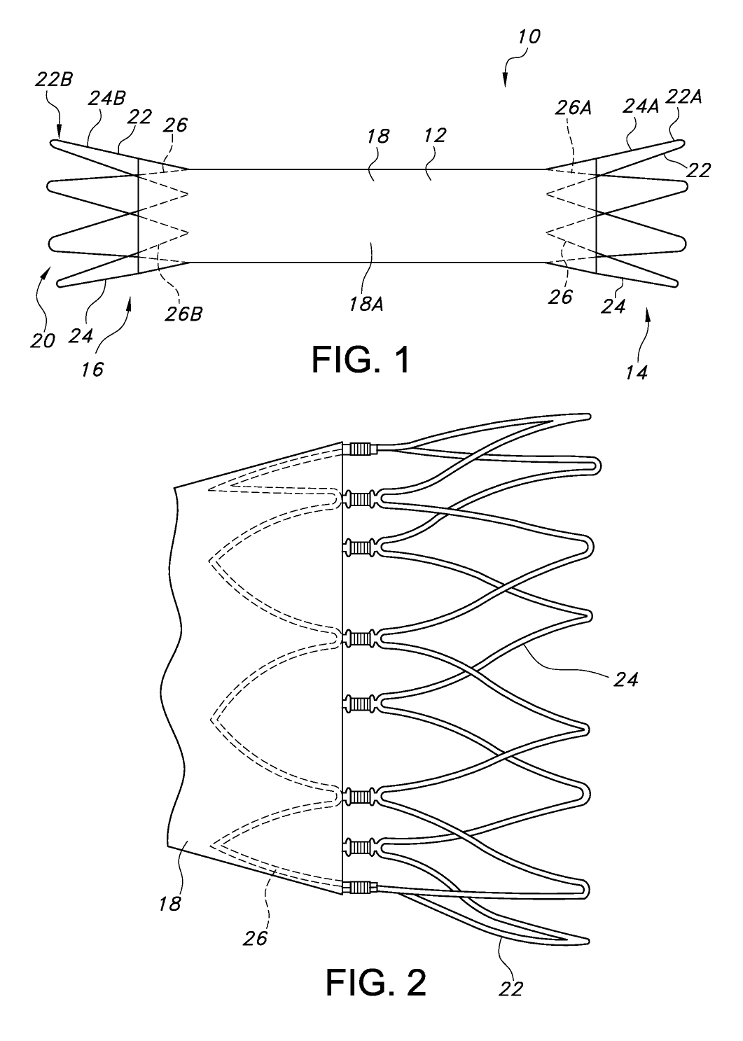

FIG. 1 depicts a sac liner assembly 10 for the treatment of an aneurysm, such as, but not limited to, a thoracic aortic aneurysm. As depicted in FIG. 1, the sac liner 10 includes a main liner member 12 disposed between a proximal open end 14 and an opposed open distal end 16. The main liner 12 has a wall portion 18 that bounds a main fluid flow lumen 20 disposed therein and between the opposed open ends 14, 16. The liner wall portion 18 may be made from any biocompatible, durable material, including, for example, polytetrafluoroethylene (“PTFE”), polyethylene terephthalate (PET″), and the like. Unless otherwise specifically stated, the term “PTFE” as used herein includes PTFE, porous PTFE and ePTFE, any of which may be impermeable, semi-permeable, or permeable. Furthermore, the sac liner assembly 10 and any portions thereof including the main body and extensions described herein may include all PTFE, all ePTFE, all porous PTFE, or any combination thereof, for example, in a single layer; more than one layer to form a composite sac liner may also be used. In one particular embodiment, the liner wall portion 18 includes a porous PTFE material having no discernable node and fibril structure. Methods of formation of such materials include those methods described in U.S. Patent Application Publication No. 2006/0233990, entitled “PTFE Layers And Methods Of Manufacturing”, which is incorporated by reference in its entirety herein. In another particular embodiment, the liner wall portion 18 includes partially sintered ePTFE material having greater flexibility or expansive properties over fully sintered ePTFE.

With regard to graft embodiments discussed herein, such as sac liner assembly 10, and components thereof, the term “proximal” refers to a location towards a patient's heart and the term “distal” refers to a location away from the patient's heart. With regard to delivery system catheters and components thereof discussed herein, the term “distal” refers to a location that is disposed away from an operator who is using the catheter and the term “proximal” refers to a location towards the operator.

The sac liner assembly 10 may include a proximal anchor member 22A, which may be disposed at a proximal end 14 of the main liner 12. One representative anchor system may include one as depicted in FIG. 2. The anchor member 22 includes a proximal stent 24, which may be self-expanding or may be balloon-expandable, that is formed from an elongate element having a generally serpentine shape with a number of crowns or apices at either end. As depicted in FIG. 2, eight crowns or apices are shown for stent 24A. The number of crowns or apices is not limiting and any suitable number may be used. A distal and/or proximal end of the stent 24 may be mechanically coupled to a connector ring 26 which is embedded in graft material, either at the proximal end 14 of the main liner 12 or at the distal end 16 of the main liner 12, or directly coupled to perforations in the proximal or distal edge region of the main liner. Embodiments of the connector ring 26 may be generally circular in shape and may have regular undulations about the circumference that may be substantially sinusoidal in shape. As depicted in FIG. 1, the proximal end 14 of the sac liner assembly 10 may include a proximal anchor member 22A. The proximal anchor member 22A may similarly include a proximal self-expanding stent 24A, which may be mechanically coupled to a proximal connector ring 26A. In addition, the sac liner assembly 10 may include a similar configuration at the distal end 16. The distal end 16 of the sac liner assembly 10 may include a distal anchor member 22B. The distal anchor member 22B may similarly include a distal self-expanding stent 24B, which may be mechanically coupled to a distal connector ring 26B. It is understood that the sac liner assembly 10 may include a proximal anchor member 22A only, a proximal anchor member 22A and a distal anchor member 22B, or neither of a proximal anchor member 22A or a distal anchor member 22B. U.S. Pat. No. 7,147,660, entitled “Advanced Endovascular Graft”, which is incorporated by reference herein in its entirety, also includes anchor member embodiments that may be used for embodiments discussed herein.

The sac liner assembly 10 is not limited to the use of connector rings for securing anchor members to the liner portions of the sac liner assembly 10. Other securing techniques and securing members, such as those disclosed in U.S. Application Publication Nos. US 2013-0268056 A1, entitled “Low Profile Stent Graft And Delivery System”, and US 2013-0268057 A1, entitled “Low Profile Stent Graft And Delivery System”, the entirety of each of which is incorporated herein by reference, may suitably be used. Since the main liner 12 can conform to the aneurysm sac, relatively low displacement forces act on the sac liner assembly 10, and thus anchor members may not be required to resist the larger displacement loads present in conventional stent graft systems. The anchor members contribute to the establishment of an acute seal at the ends of the main liner 12, so as to facilitate apposition of the liner 12 to the sac wall. Alternative means of sealing, including the use of inflatable annular rings as described in the above references, can be employed as well.

Anchor member 22 may be configured as a self-expanding anchor member having an undulating pattern and may be made from stainless steel, nickel titanium alloy or any other suitable material. The anchor member 22 may be configured to be balloon expandable or self-expanding in an outward radial direction from a radially compressed state. The proximal anchor member 22 and its components may have the same or similar features, dimensions or materials to those described in U.S. Pat. Nos. 7,147,660 and 6,395,019, the content of each of which is hereby incorporated by reference herein in its entirety.

Various methods of delivery systems and delivery of the device into a patient include those described in Applicant's application, U.S. Patent Application Publication No. 2009/0099649, entitled “Modular Vascular Graft For Low Profile Percutaneous Delivery”, the contents of which are incorporated by reference in its entirety herein. For endovascular methods, access to a patient's vasculature may be achieved by performing an arteriotomy or cut down to the patient's femoral artery or by other common techniques, such as the percutaneous Seldinger technique. For such techniques, a delivery sheath (not shown) may be placed in communication with the interior of the patient's vessel such as the femoral artery with the use of a dilator and guidewire assembly. Once the delivery sheath is positioned, access to the patient's vasculature may be achieved through the delivery sheath which may optionally be sealed by a hemostasis valve or other suitable mechanism. For some procedures, it may be necessary to obtain access via a delivery sheath or other suitable means to both femoral arteries of a patient with the delivery sheaths directed upstream towards the patient's aorta. In some applications a delivery sheath may not be needed and a delivery catheter may be directly inserted into the patient's access vessel by either arteriotomy or percutaneous puncture.

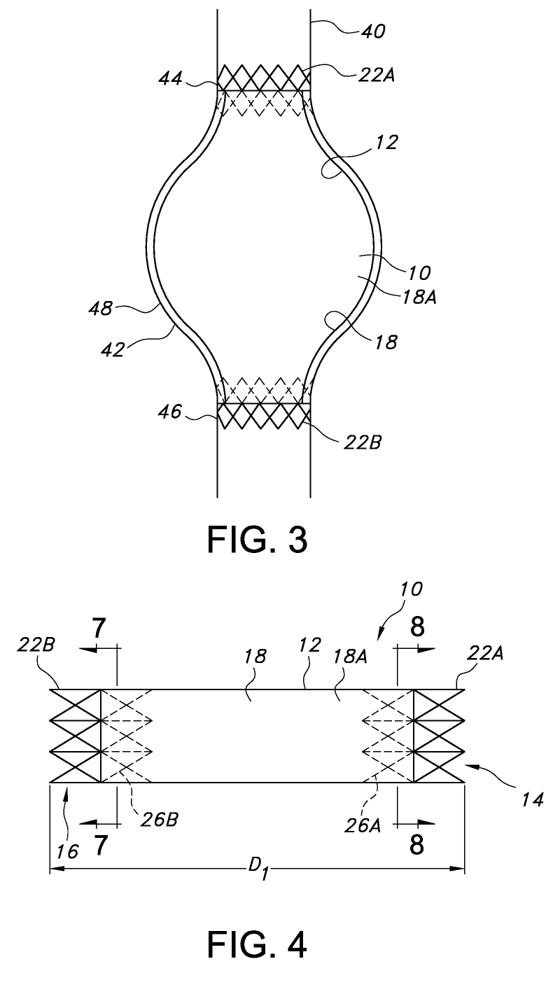

FIG. 3 depicts deployment of the sac liner assembly 10 within an aneurysm 42, such as a TAA or iliac aneurysm, of an artery 40. The proximal anchor member 22A may be may be disposed at a proximal location 44 (or upstream) of the aneurysm 42. The distal anchor member 22B may be disposed at a distal location 46 (or downstream) of the aneurysm 42. The liner wall portion 18 of the sac liner assembly 10 is flexible and/or expansive to substantially conform to the shape of the aneurysmal wall 44. In conforming to the shape of the aneurysmal wall 48, the liner wall portion 18 may contact portions, including substantially all of the portions of the aneurysmal wall 48. As such the liner wall portion 18 reinforces or strengthens the aneurysmal wall 44, in contrast to effectively bypassing the aneurysmal wall with known stent-grafts having substantially tubular flow lumens. Effectively strengthening the aneurysmal wall 48 is important because studies have reported that aneurysmal wall tissue is weaker than non-aneurysmal wall tissue or normal arterial wall tissue. For example, Vorp, David A., PhD, “Biomechanics of Abdominal Aortic Aneurysm”, J. Biomed., Vol. 40(9), 2007, 1187-1902, reports that aneurysmal wall tissue may be about 40 t0 50 percent weaker than normal arterial wall tissue. Additionally, the present invention recognizes that by utilizing the strength of aneurysmal wall tissue, even though it is reduced as compared to normal arterial wall tissue, a lower profile sac liner assembly may be used as compared to known EVAR devices and assemblies. Moreover, the liner wall portion 18 of the sac liner assembly 10 can also accommodate the higher aneurysmal wall stresses typically associated with aneurysms. For example, it has been reported that normal stresses in an undilated aorta may vary from about 5 to 12 N/cm² as compared to stresses of up to 40 N/cm² in portions of the aneurysmal itself. (See, e.g., Vorp, supra).

To provide low profile while still providing sufficient augmenting strength to the aneurysmal wall 48, the liner wall portion 18 may be a single layer of polymeric membrane material or a laminated composite structure comprising two or more polymeric membranes. In one embodiment, the polymeric membrane material includes PTFE which is substantially porous but includes no discernable node and fibril structure. The liner wall portion 18 may be formed from tubular extrusions, laminated wraps of single of multiple laminated layers of membrane material, and the like, in any desirable combination. The polymeric membrane material may be permeable, semi-permeable or substantially non-permeable for some embodiments. For embodiments that include laminated wraps of material, the wraps may be carried out circumferentially, helically or in any other suitable configuration. For some embodiments, the liner wall portion 18 may be a layer of partially sintered ePTFE. The thickness of the polymeric membrane material may vary from about 0.0001 inches (or about 0.1 mils) to about 0.002 inches (or about 2 mils). More typically, the thickness of the polymeric membrane material may vary from about 0.00045 inches (or about 0.45 mils) to about 0.0012 inches (or about 1.2 mils). If multiple membrane layers are used, the thickness of the laminated polymeric membrane materials may vary from about 0.0028 inches (or about 2.8 mils) to about 0.0085 inches (or about 8.5 mils). Additionally, useful membrane or wall thicknesses may include thicknesses from about 0.005 inches or less than about 0.005 inches; from about 0.0012 inches or less than about 0.0012 inches; from about 0.0007 inches or less than about 0.0007 inches; and from about 0.0005 inches or less than about 0.0005 inches. Such membrane or wall thickness are non-limiting and any suitable membrane or wall thicknesses may be used provided that the liner wall portion 18 may flexibly and/or expansively conform to the aneurysmal wall 48 upon deployment.

The medial liner wall portion 18A may be a unitary liner wall not having spaced-apart layers or membranes thereat. Further, in one embodiment, the medial liner wall portion 18A does not have re-enforcing filaments, strands or stent portions thereat. The medial liner wall portion 18A may comprise a layer of extruded, non-textile polymeric material or may comprise a plurality of layers of extruded, non-textile polymeric material sintered together to provide the unitary liner wall. As used herein, the term “textile” refers to a material, such as a filament or yarn, that has been knitted, woven, braided and the like into a structure, including a hollow, tubular structure. As used herein, the term “non-textile” and its variants refer to a material formed by casting, molding, spinning or extruding techniques to the exclusion of typical textile forming techniques, such as braiding, weaving, knitting and the like.

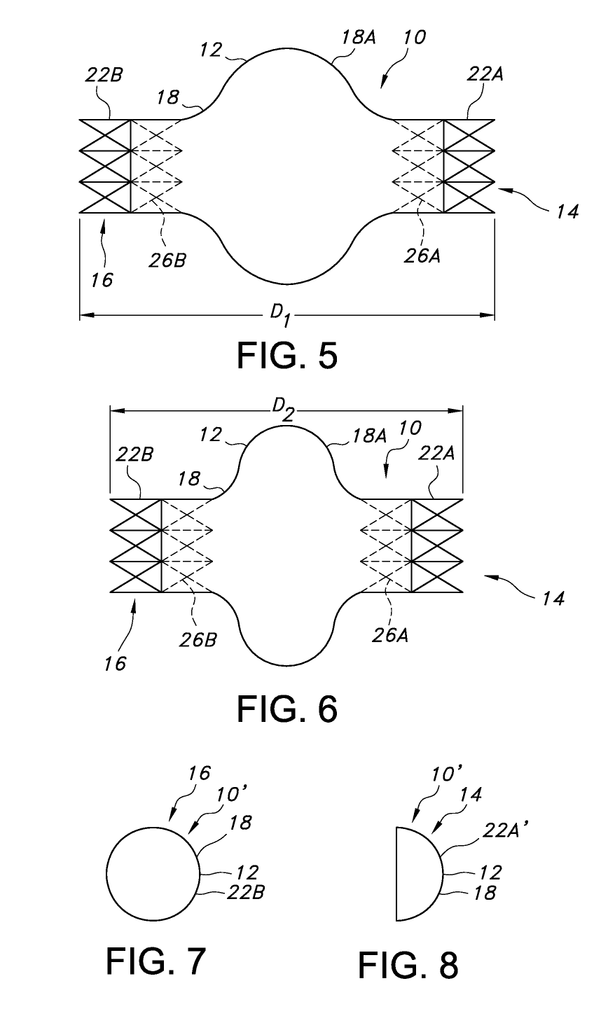

FIGS. 4-6 depict embodiments of the sac liner assembly 10 of the present invention. In FIG. 4, the sac liner assembly 10 is depicted in one embodiment in its quiescent form not being subject to arterial blood pressure. The liner wall portion 18, 18A is depicted as being substantially tubular. The present invention, however, is not so limited. For example, as depicted in FIG. 5, the liner wall portion 18, 18A of the sac liner assembly 10 may have a bulbous shape in its quiescent state. In comparing FIGS. 4 and 5 the overall length of the sac liner assembly 10 is about the same; i.e., D₁. During deployment (not shown), the sac liner assembly 10 of FIG. 5 may be compressed to a reduced profile by compressing the anchor members 22A, 22B and by moving the anchor members 22A, 22B longitudinally away from one and the other to compress the bulbous shape depicted in FIG. 5 to a lower profile shape, such as the shape depicted in FIG. 4; i.e., where the liner wall portion 18, 18A does not extend significantly past the anchor members 22A, 22B in a radial or circumferential direction.

The liner wall 18 of the medial liner portion 18A is flexibly expansive to conform to a wall 48 of the aneurysm 42 to provide additional strength to aneurysmal tissue at the aneurysm wall 48. The liner wall 18 of the medial liner portion 18A may be flexible to expand to a bulbous shape to substantially conform to the shape of the aneurysmal wall 48. The liner wall 18 of the medial liner portion 18A may be crimped or partially folded in its quiescent state to further provide flexibility from its quiescent state to its deployed shape. After deployment, the sac liner assembly 10 inhibits further growth in diameter of the aneurysm 42, including substantially inhibiting further growth in diameter of the aneurysm 42 and including at least partially inhibiting further growth in diameter of the aneurysm 42. After deployment, the sac liner assembly 10 inhibits failure of the aneurysmal wall 48, including substantially inhibiting failure of the aneurysmal wall 48.

In another embodiment, as depicted in FIG. 6, the anchor members 22A, 22B may be disposed closer to one and the other upon deployment as indicated by D₂ as compared to quiescent longitudinal spacing D₁ shown in FIG. 4. Such deployed closer spacing of the anchor members 22A, 22B and the arterial blood pressure pressing onto the liner wall portion 18 after the proximal open end 14 and the open distal end 16 of the sac liner assembly 10 are securably and sealing disposed proximally and distally, respectively, of an aneurysm may also facilitate the liner wall portion 18 in achieving a bulbous shape which substantially conforms and contacts all or substantially all of the aneurysmal wall tissue.

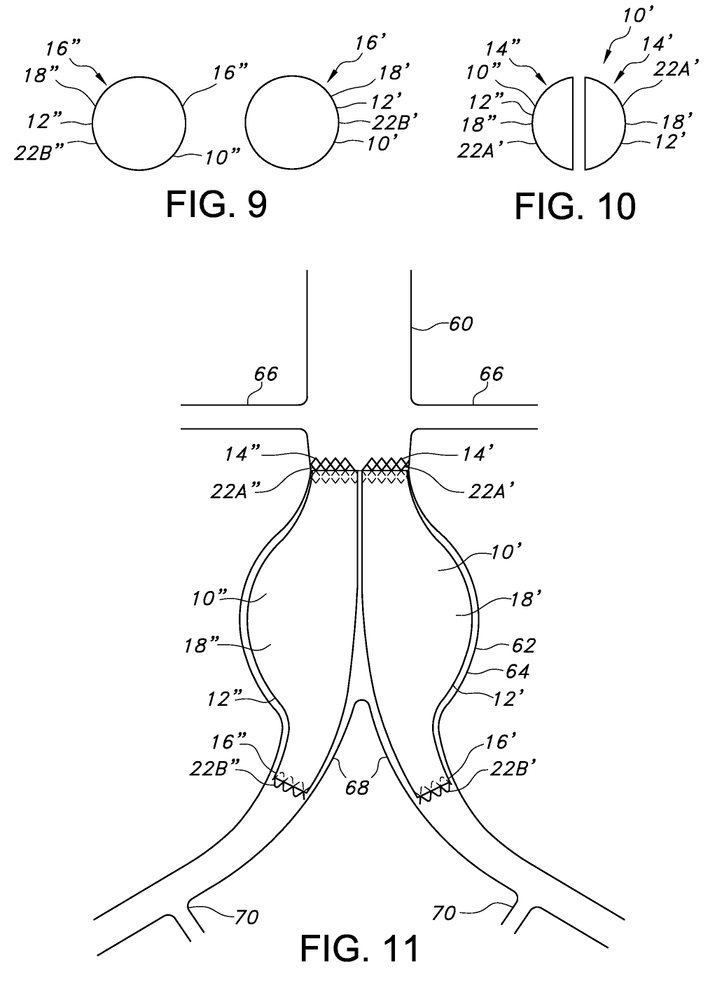

The present invention is not limited to the treatment and management of aneurysm in single lumens or arteries. For example, as depicted in FIGS. 7 through 11, the sac liner assembly 10 or assemblies 10′, 10″ may suitably be used to treat and manage aneurysms at branched lumens or arteries, such as, such as abdominal aortic aneurysms. For example, as depicted in FIG. 7 the distal end 16 or the sac liner assembly 10 may have a tubular or substantial tubular or circular shape while, as depicted in FIG. 8, the proximal end 14 of the sac liner assembly 10 may have a non-tubular or non-circular shape. In one embodiment as depicted in FIG. 8, the proximal end 14 of the sac liner assembly 10 may have a D-shape. As depicted in FIGS. 9-10, pairs of sac liner assemblies 10′, 10″ having D-shaped proximal ends 14′, 14″ may be used in concert with one and the other.

FIG. 11 depicts pairs of sac liner assemblies 10′, 10″ in a deployed state within a bodily lumen, for example, an aorta 60. The main liner member 12′ of the sac liner assembly 101 and the main liner member 12″ of the sac liner assembly 10″ span or substantially span the diseased region of abdominal aorta or aneurysm 62. The proximal self-expanding stent 24A′ of the sac liner assembly 10′ and the proximal anchor member 22A″ of the sac liner assembly 10″ are disposed proximally (or above) relative to the aneurysm 62. As depicted in FIG. 11, the proximal self-expanding stent 24A′ of the sac liner assembly 10′ and the proximal anchor member 22A″ of the sac liner assembly 10″ are disposed distally (or below) relative to the renal arteries 66. The present invention, however, is not so limited. For example, the proximal self-expanding stent 24A′ of the sac liner assembly 10′ and the proximal anchor member 22A″ of the sac liner assembly 10″ may be disposed across the renal arteries 66. Such a placement of the proximal anchor members 22A′, 22A″ may be desirable where the aneurysm 62 is close to the renal arteries 66. In such a placement the extent of length of the proximal anchor members 22A′, 22A″ should be sufficient such that they span the renal arteries 66 while the main liner members 12′, 12″ do not span the renal arteries 66.

The distal anchor member 22B′ of the sac liner assembly 10′ and the distal anchor member 22B″ of the sac liner assembly 10″ are disposed distally (or below) the aneurysm 62. The distal anchor members 22B′, 22B″ are deployed in the iliac arteries 68 above the hypogastric arteries 70.

The liner wall portion 18′ of the sac liner assembly 10′ and the liner wall portion 18″ of the sac liner assembly 10″ substantially conform and/or contact the aneurysmal wall 64 of the aneurysm 62 to provide additional strength to the aneurysmal wall 64. With the proximal end 14′ of the sac liner assembly 10′ and the proximal end 14″ sac liner assembly 10″ sealingly engaging the aorta 60 and with the distal end 16′ of the sac liner assembly 10′ and the distal end 16″ of the sac liner assembly 10″ sealingly engaging the iliac arteries 68, the liner wall portions 18′, 18″ effectively prevent endoleaks, including Type II endoleaks.

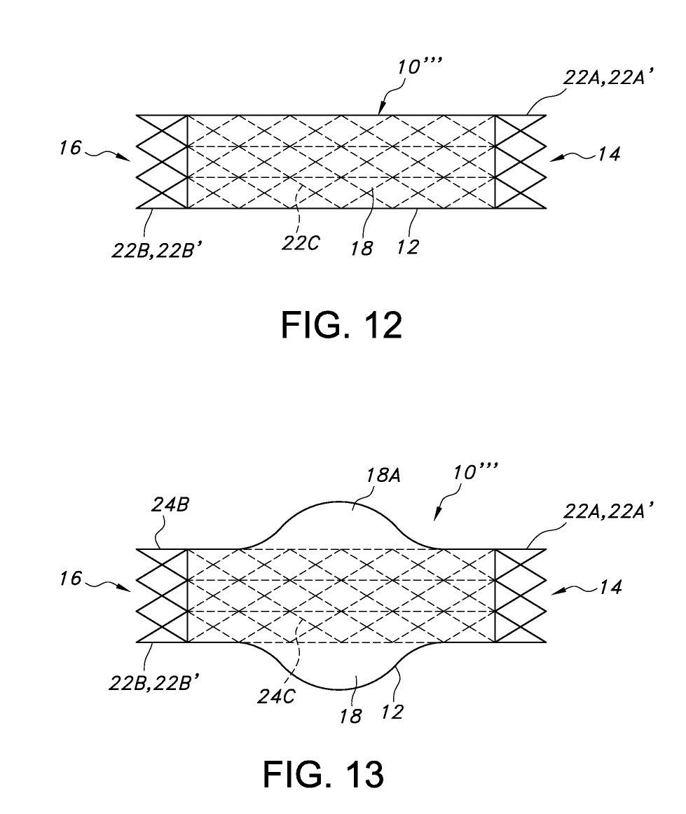

FIGS. 12 and 13 depict another embodiment of the sac liner assembly 10′″ of the present invention. The sac liner assembly 10′″ includes a medial stent 24C disposed between the proximal stent 24A and the distal stent 24B. The stents 24A, 24B, 24C may be unitary or may be modular. The stents 24A, 24B, 24C may be formed from an elongate resilient element helically wound with a plurality of longitudinally spaced turns into an open tubular configuration. The helically wound stents 24A, 24B, 24C may be configured to be a self-expanding stent or radially expandable in an inelastic manner actuated by an outward radial force from a device such as an expandable balloon or the like. The stents 24A, 24B, 24C may be formed from a plurality of elongate resilient elements helically wound, braided or knotted into the open tubular configuration. Some tubular prosthesis embodiments that may be used for the self-expanding stents 24A, 24B, 24C are discussed in U.S. Pat. No. 6,673,103, entitled “Mesh and Stent for Increased Flexibility”, which is hereby incorporated by reference in its entirety herein.

The liner wall portion 18 is not secured to the medial stent 24C portion. The liner wall portion 18 of the sac liner assembly 10′″ is flexibly expansive so that upon deployment of the sac liner assembly 10′″, the liner wall portion 18 conform and/or contacts the aneurysmal wall. The nominal blood flow lumen may be acutely maintained by the bare stent structure of the medial stent 24C, and the space exterior to the medial stent 24C may nominally fill and clot over time. The degree of clotting, if desired, may depend on the porosity of the medial stent 24C, flow configuration of the sac liner assembly 10′″, etc.

While various embodiments of the present invention are specifically illustrated and/or described herein, it will be appreciated that modifications and variations of the present invention may be effected by those skilled in the art without departing from the spirit and intended scope of the invention. Further, any of the embodiments or aspects of the invention as described in the claims or in the specification may be used with one and another without limitation.

What is claimed is:

1. An endovascular system for aneurysm management comprising:
a vascular liner having a proximal open end at a proximal liner portion and an opposed distal open end at a distal liner portion defining an open liner lumen having a liner wall disposed there between and further defining a medial liner portion disposed between the proximal liner portion and the distal liner portion;
a proximal anchor associated with the proximal liner portion; and
a distal anchor associated with the distal liner portion;
wherein the liner wall of the medial liner portion is an unitary liner wall not having spaced-apart layers or membranes thereat and not having re-enforcing filaments, strands or stent portions thereat; and
wherein the liner wall of the medial liner portion is flexibly expansive to conform to a wall of an aneurysm to provide additional strength to aneurysmal tissue at the aneurysm wall.

2. The endovascular system of claim 1, wherein the liner wall of the medial liner portion comprises an extruded, non-textile polymeric material.

3. The endovascular system of claim 2, wherein the extruded, non-textile polymeric material comprises a material selected from the group consisting of polytetrafluoroethylene, expanded polytetrafluoroethylene having a node and fibril structure, expanded porous polytetrafluoroethylene not having a node and fibril structure, and combinations thereof.

4. The endovascular system of claim 2, wherein the liner wall of the medial liner portion comprises a plurality of layers of extruded, non-textile polymeric material laminated together to provide the unitary liner wall.

5. The endovascular system of claim 3, wherein the liner wall of the medial liner portion comprises a plurality of layers of extruded, non-textile polymeric material sintered together to provide the unitary liner wall.

6. The endovascular system of claim 1, wherein the liner wall of the medial liner portion has a wall thickness of about 0.005 inches or less than about 0.005 inches.

7. The endovascular system of claim 1, wherein the liner wall of the medial liner portion has a wall thickness of about 0.0012 inches or less than about 0.0012 inches.

8. The endovascular system of claim 1, wherein the liner wall of the medial liner portion has a wall thickness of about 0.0007 inches or less than about 0.0007 inches.

9. The endovascular system of claim 1, wherein the liner wall of the medial liner portion has a wall thickness of about 0.0005 inches or less than about 0.0005 inches.

10. The endovascular system of claim 1, wherein the liner wall of the medial liner portion is flexible to expand to a bulbous shape to substantially conform to the shape of the aneurysmal wall.

11. The endovascular system of claim 1, wherein the liner wall of the medial liner portion has a bulbous shape to generally conform to the shape of the aneurysmal wall.

12. The endovascular system of claim 1, wherein the liner wall of the medial liner portion is crimped.

13. The endovascular system of claim 1, wherein the proximal anchor is securably disposed at the proximal liner portion.

14. The endovascular system of claim 1, wherein the proximal anchor is securably affixed to the proximal liner portion.

15. The endovascular system of claim 1, wherein the distal anchor is securably disposed at the distal liner portion.

16. The endovascular system of claim 1, wherein the distal anchor is securably affixed to the distal liner portion.

17. The endovascular system of claim 1, further comprising:
a medial stent disposed between the proximal and distal anchor;
wherein the liner wall of the medial liner portion is not securably affixed to substantial portions of the medial stent.

18. The endovascular system of claim 1, wherein the proximal liner portion is D-shaped.

19. The endovascular system of claim 18, further comprising:
a second vascular liner having a proximal open end at a proximal liner portion and an opposed distal open end at a distal liner portion defining an open liner lumen having a liner wall disposed there between and further defining a medial liner portion disposed between the proximal liner portion and the distal liner portion;
a second proximal anchor associated with the proximal liner portion of the second vascular liner; and
a second distal anchor associated with the distal liner portion of the second vascular liner;
wherein the liner wall of the medial liner portion of the second vascular liner is an unitary liner wall not having spaced-apart layers or membranes thereat and not having re-enforcing filaments, strands or stent portions thereat;
wherein the liner wall of the medial liner portion of the second vascular liner is flexibly expansive to conform to a wall of an aneurysm to provide additional strength to aneurysmal tissue at the aneurysm wall to inhibit failure of the aneurysm wall;
wherein the proximal liner portion of the second vascular liner is D-shaped; and
wherein the D-shaped portion of the vascular liner and the D-shaped portion of the second vascular liner are complimentary such that the proximal liner portions of the vascular liner and the second vascular liner are deployable within a main artery proximal to the aneurysm, the distal portion of the vascular liner is deployable with a first branched artery distal of the aneurysm and the distal portion of the second vascular liner is deployable with a second branched artery distal of the aneurysm.

20. A method for treating an aneurysm comprising:
providing a vascular system comprising:
a vascular liner having a proximal open end at a proximal liner portion and an opposed distal open end at a distal liner portion defining an open liner lumen having a liner wall disposed there between and further defining a medial liner portion disposed between the proximal liner portion and the distal liner portion;
a proximal anchor associated with the proximal liner portion; and
a distal anchor associated with the distal liner portion;
wherein the liner wall of the medial liner portion is an unitary liner wall not having spaced-apart layers or membranes thereat and not having re-enforcing filaments, strands or stent portions thereat; and
deploying the vascular liner system such that the proximal anchor is disposed proximally beyond an aneurysm and such that the distal anchor is disposed distally beyond the aneurysm; and
expanding the liner wall of the medial liner portion to allow the liner wall of the medial liner portion to conform to a wall of an aneurysm to provide additional strength to aneurysmal tissue at the aneurysm wall.

21. The method of claim 20, wherein, after the liner wall of the medial liner portion is deployed to the wall of the aneurysm, the vascular system inhibits failure of the aneurysmal wall.

22. The method of claim 20, wherein, after the liner wall of the medial liner portion is deployed to the wall of the aneurysm, the vascular system inhibits further growth in diameter of the aneurysm.

23. The method of claim 20, wherein the proximal liner portion is D-shaped.

24. The method of claim 23, further comprising
providing a second vascular liner having a proximal open end at a proximal liner portion and an opposed distal open end at a distal liner portion defining an open liner lumen having a liner wall disposed there between and further defining a medial liner portion disposed between the proximal liner portion and the distal liner portion;
providing a second proximal anchor associated with the proximal liner portion of the second vascular liner; and
providing a second distal anchor associated with the distal liner portion of the second vascular liner;
wherein the liner wall of the medial liner portion of the second vascular liner is an unitary liner wall not having spaced-apart layers or membranes thereat and not having re-enforcing filaments, strands or stent portions thereat;
wherein the proximal liner portion of the second vascular liner is D-shaped; and
deploying the D-shaped portion of the vascular liner and the D-shaped portion of the second vascular liner within a main artery proximal to the aneurysm such that the D-shaped portions are complimentary to provide flow paths of blood through the vascular liner and the second vascular liner without a substantial flow path of blood to the aneurysm;
deploying the distal portion of the vascular liner within a first branched artery distal of the aneurysm;
deploying the distal portion of the second vascular liner within a second branched artery distal of the aneurysm; and
expanding the liner wall of the medial liner portion of the second vascular liner to allow the liner wall of the medial liner portion of the second vascular liner to conform to a wall of the aneurysm to provide additional strength to aneurysmal tissue at the aneurysm wall.

25. An endovascular system for aneurysm management comprising:
a vascular liner having a proximal open end at a proximal liner portion and an opposed distal open end at a distal liner portion defining an open liner lumen having a liner wall disposed there between and further defining a medial liner portion disposed between the proximal liner portion and the distal liner portion;
wherein the liner wall of the medial liner portion is an unitary liner wall not having spaced-apart layers or membranes thereat and not having re-enforcing filaments, strands or stent portions thereat; and
wherein the liner wall of the medial liner portion is flexibly expansive to conform to a wall of an aneurysm to provide additional strength to aneurysmal tissue at the aneurysm wall.

26. The endovascular system of claim 25, further comprising:
a proximal anchor associated with the proximal liner portion; and
a distal anchor associated with the distal liner portion;
